(19) United States
(12) Patent Application Publication
(10) Pub. No.: US 2025/0241547 A1
(43) Pub. Date: Jul. 31, 2025

(54) METHODS AND SYSTEMS FOR POST-OPERATIVE PATIENT CONDITION PREDICTION

(71) Applicant: GE Precision Healthcare LLC, Waukesha, WI (US)

(72) Inventors: Emilia Elli-Noora Robertson, Helsinki (FI); Mika Olli Kristian Särkelä, Helsinki (FI)

(21) Appl. No.: 18/424,547

(22) Filed: Jan. 26, 2024

(51) Int. Cl. A61B 5/024; A61B 5/00; A61B 5/02; A61B 5/1455

(52) U.S. Cl. CPC A61B 5/02416; A61B 5/02028; A61B 5/14551; A61B 5/7239; A61B 5/7275; A61B 5/742

(57) ABSTRACT

Described herein are methods and systems for predicting a post-operative condition that may be experienced by a patient, following a surgical procedure. In one or more embodiments, a method for quantifying a patient response to surgery comprises generating a single index that quantifies a patient condition of a patient using a nociception response of the patient during a surgical procedure, where the nociception response includes a hemodynamic response of the patient detected within a non-fixed length, continuously increasing window of time that increases until conclusion of the surgical procedure.

### TECHNICAL FIELD

Embodiments of the subject matter disclosed herein relate to patient monitoring, and more particularly, to predicting a post-operative condition of a patient using nociception responses of the patient.

### BACKGROUND

Patient monitors routinely process signals acquired from patients and provide a caregiver or clinician with computed estimates of features contained within those signals. Patient monitors may be invasive or non-invasive, and be configured to monitor one or more of blood pressure, pulse-oximetry, temperature, carbon dioxide, respiration, cardiac output, and so on. In the case of electrocardiogram (ECG) signals, features include heart rate and arrhythmias (e.g., disturbances in normal cardiac rhythm). In the case of pulse-oximetry signals, features include saturation of oxygen in a patient's blood.

Activation of the sympathetic nervous system of the patient, as a result of multiple stimuli and inputs experienced by the patient during a surgical procedure, leads to multiple nociception-related physiological responses, which may be captured by one or more patient monitors. Features monitored by a patient monitor may be used to objectively quantify a patient's physiological condition during a surgical procedure, which may enable medical providers to personalize pain relief treatment. A patient condition may include, for example, a physical pain response (e.g., nociception), and a mental state of the patient (e.g., state of delirium). A patient monitoring system may quantify information from multiple features measured by one or more patient monitors as a nociception level. For example, the nociception level may be determined using measurements of cortical responses (e.g., pain and other sensations), heart rate variability, and other physiological responses to painful/noxious stimulus. The nociception level may be used by medical providers, such as anesthesiologists, to guide use of opioids during and after the surgical procedure, which may assist in decreasing a post-operative pain level of the patient.

### SUMMARY

In one embodiment, a method for quantifying a patient response to surgery comprises generating a single index that quantifies a patient condition of a patient using a nociception response of the patient during a surgical procedure, where the nociception response includes a hemodynamic response of the patient detected within a non-fixed length, continuously increasing window of time that increases until conclusion of the surgical procedure. The single index is generated based on a hemodynamic response detected within a defined time period. The defined time period is a non-fixed length, continuously increasing window of time that increases until the conclusion of the surgical procedure. The method may be executed by a patient monitoring system comprising a patient monitor, a display device, and a computing device communicably coupled to the patient monitor and the display device. The computing device is configured to continuously obtain a hemodynamic response of a patient as photoplethysmogram signal data including a photoplethysmogram signal amplitude (PPGA) and/or a heartbeat interval (HBI). Derivatives and statistical features may be derived from the photoplethysmogram signal data and used to quantify hemodynamic responses by combining statistical features to obtain a first index and a second index. The first index (e.g., the above mentioned single index) is an aggregate value of statistical features of HBI time series data, statistical features of derivatives of PPGA time series data, and statistical features of derivatives of normalized HBI time series data during a first time period, where the first time period is a continuously growing time window. In this context, the statistical features are measures characterizing data values and distribution, such as mean, standard deviation, skewness, kurtosis and percentiles, for example. The second index is a combination of normalized HBI time series data and normalized PPGA time series data during a second time period, where the second time period is a fixed-length, sliding time window that is shorter than and included in the first time period. In some embodiments, in response to the first index exceeding a nociception threshold, the method may further include outputting instructions to administer analgesic medication.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 shows a block diagram illustrating an example patient monitoring system according to an embodiment of the disclosure;

FIG. 2 shows an example interface of a display device of the patient monitoring system of FIG. 1 during patient monitoring;

FIG. 3 shows a series of graphs illustrating performance of a method for quantifying a patient condition following conclusion of a surgical procedure using data captured from a patient not experiencing a hemodynamic response;

FIG. 4 shows a series of graphs illustrating performance of the method for quantifying the patient condition using data captured from a patient experiencing a hemodynamic response;

FIG. 5 shows a graph of Shapley additive explanation (SHAP) values of statistical features used to train the method for quantifying the patient condition;

FIG. 6 shows a graph of SHAP values of statistical features used to test the method for quantifying the patient condition;

FIG. 7 shows a series of graphs illustrating hemodynamic response of a patient as photoplethysmogram signal data, and derivatives and statistical features thereof;

FIGS. 8A and 8B show a high-level flow chart of a method for quantifying the patient condition, according to an embodiment of the disclosure;

FIG. 9 shows a high-level flow chart illustrating a method for generating a single index that characterized patient responses over a defined time period, according to an embodiment of the disclosure; and

FIGS. 10A and 10B show a flow chart illustrating a method for generating a first index defining a patient hemodynamic response over a first time period and generating a second index defining the patient hemodynamic response over a second time period, according to an embodiment of the disclosure.

The drawings illustrate specific aspects of the described systems and methods. Together with the following description, the drawings demonstrate and explain the structures, methods, and principles described herein. In the drawings, the size of components may be exaggerated or otherwise modified for clarity. Well-known structures, materials, or operations are not shown or described in detail to avoid obscuring aspects of the described components, systems and methods.

### DETAILED DESCRIPTION

This description and embodiments of the subject matter disclosed herein relate to methods and systems for quantifying a patient condition following a surgical procedure using data from a nociception response of the patient during the surgical procedure. Conventional patient monitoring systems identify a nociception level based on physiological responses to surgical nociceptive stimulus that are captured by one or more patient monitors. A nociception (e.g., pain) level may be calculated using instantaneous data (e.g., within a past five minutes) captured by the one or more patient monitors. Thus, the nociception level may characterize how a patient is doing in real-time or near-real time (e.g., within the past five minutes), and may not characterize a pain level of the patient throughout the surgical procedure. In some embodiments, such as an analgesia nociception index (ANI) parameter, an instantaneous ANI value and an average ANI value may be calculated, where the average ANI value is calculated from a sliding, fixed-length window of time (e.g., an average of multiple instantaneous ANI values).

In another example of a conventional patient monitoring system, a surgical plethysmograph index (SPI) may be used to characterize patient responses to surgical nociceptive stimulus. The SPI may be a dimensionless score based on photoplethysmograph (PPG) analysis of pulse wave and heart beat intervals (HBI), for example, as captured by a PPG sensor configured to measure volumetric variations in blood circulation. The PPG analysis provides insight into a hemodynamic response of the patient to surgical nociceptive stimuli. Values of the SPI index may be used as an indicator of post-operative pain (POP) experienced by the patient, and to trigger administration of analgesic medication. For example, a SPI value of greater than 50 may be used as a trigger to administer analgesic medication. However, in some patient cases, a baseline level (e.g., mean) SPI value after a surgical procedure may be a predictor of POP, and a significantly lower SPI value (e.g., 30) may be a more realistic cutoff between no-pain and pain.

The methods and systems described herein for quantifying a patient condition following a surgical procedure using a nociception response of the patient during the surgical procedure include providing one or more nociception levels as an indicator or a summary of the patient's pain levels throughout a defined time period (e.g., a duration of the surgical procedure), based on hemodynamic responses detected within the defined time period. The defined time period may be significantly longer than five minutes, and thus the nociception level may provide more information about the patient's condition. The method described herein uses the same physiological signals used by the SPI index, but may not use SPI cutoff values to predict POP. In one embodiment, the method comprises providing a single index that defines an overall nociception response by a patient during a surgical procedure and quantifies a patient condition following conclusion of the surgical procedure (e.g., after admittance to a post-anesthesia care unit (PACU)). The single index is generated based on a hemodynamic response detected within a defined time period, wherein the defined time period is a non-fixed length, continuously increasing window of time that increases until the conclusion of the surgical procedure. As further described herein, quantifying the patient condition may include predicting POP experienced by the patient. In some embodiments, the method includes generating a second index that defines the patient hemodynamic response over a second time period, where the second time period is a fixed-length, sliding time window that is shorter than and included in the defined time period. In further embodiments, the method may include, in response to the single index (e.g., a first index) exceeding a nociception threshold, outputting instructions to administer analgesic medication to the patient. In some embodiments, the methods described herein may be used together with SPI to provide an indicator of instantaneous patient responses (e.g., via a SPI value) and an indicator of total surgical stress experienced by a patient (e.g., summarization of multiple responses over the defined time period by the single index). In this way, medical providers may use a combination of the first index and the second index for both acute (e.g., real-time or near-real time) and preventive pain treatment (e.g., post-operative).

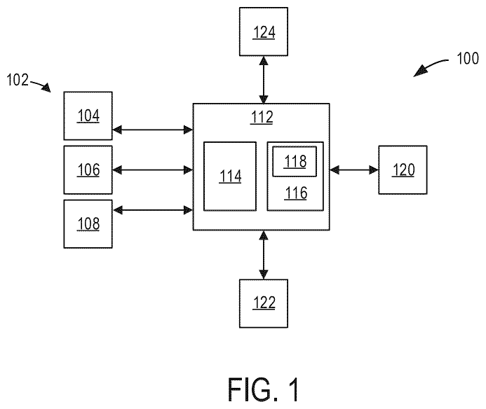

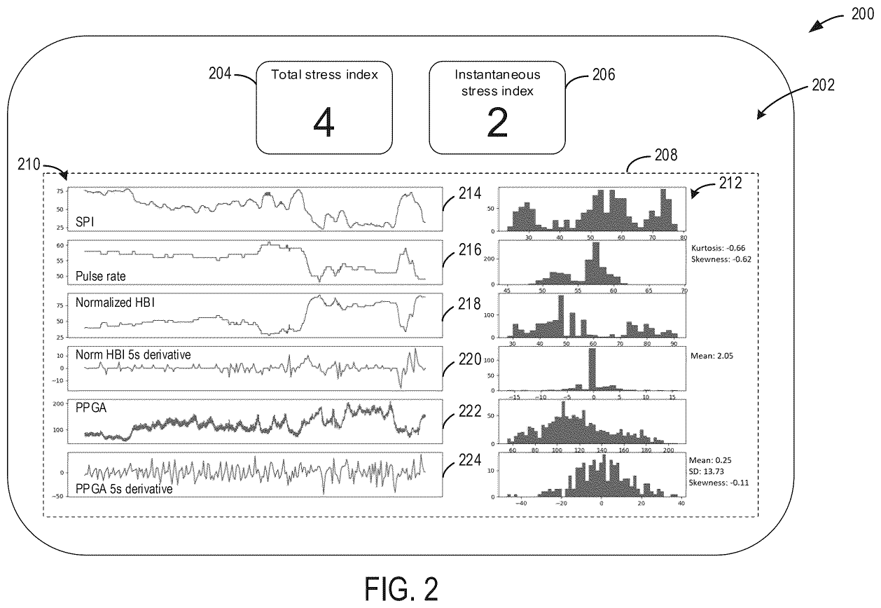

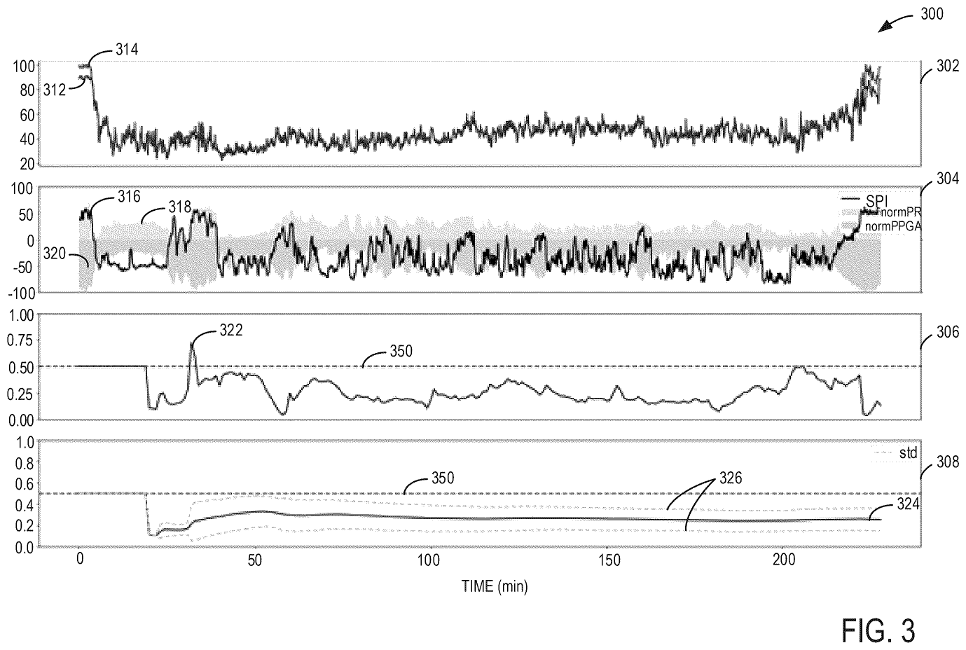

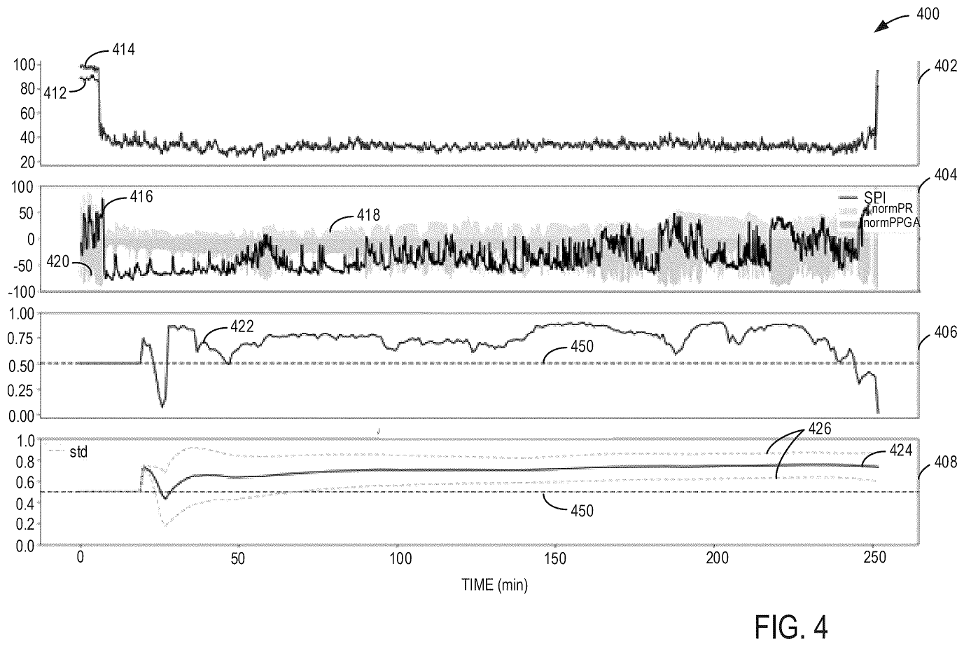

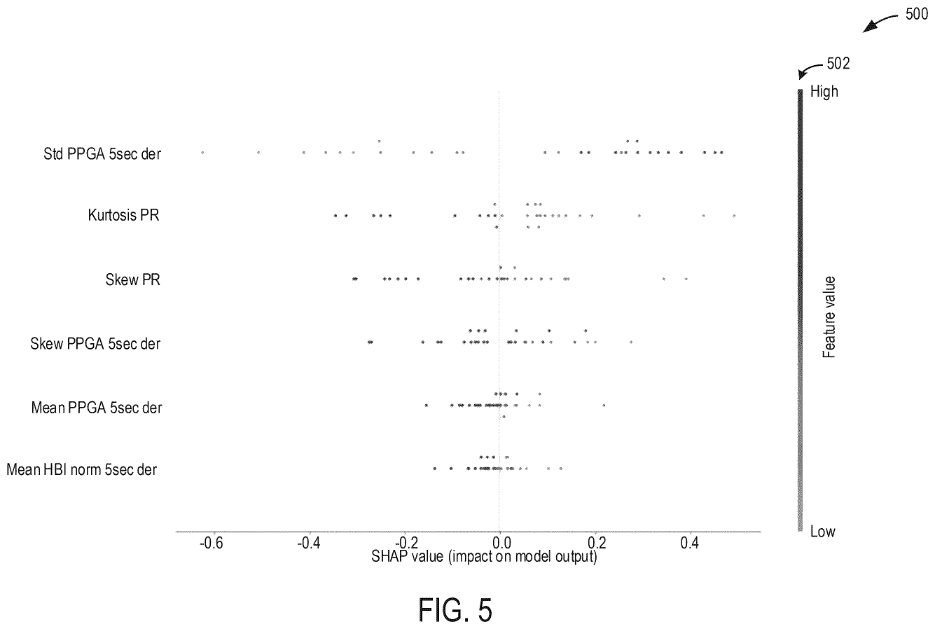

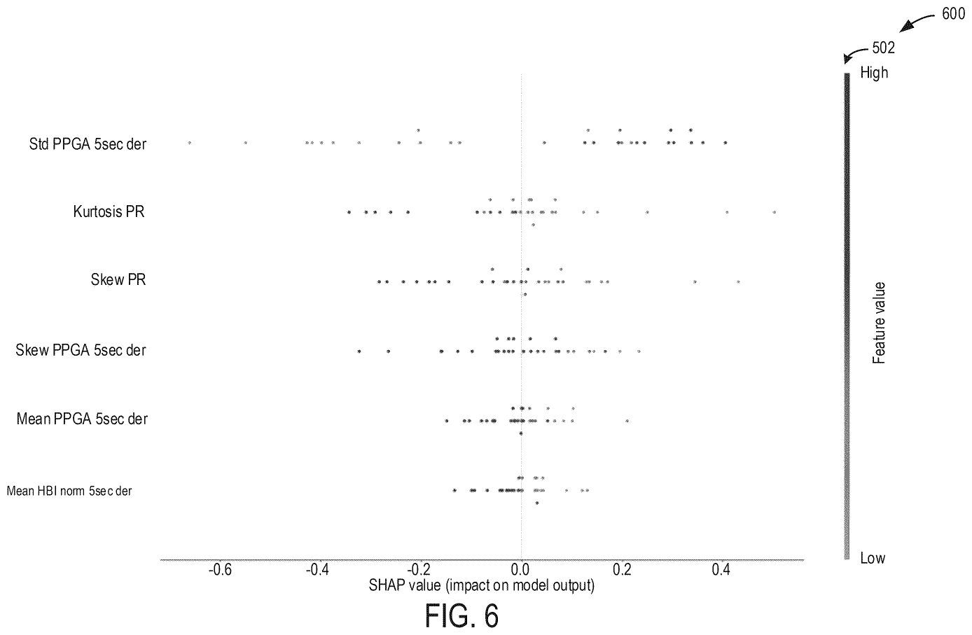

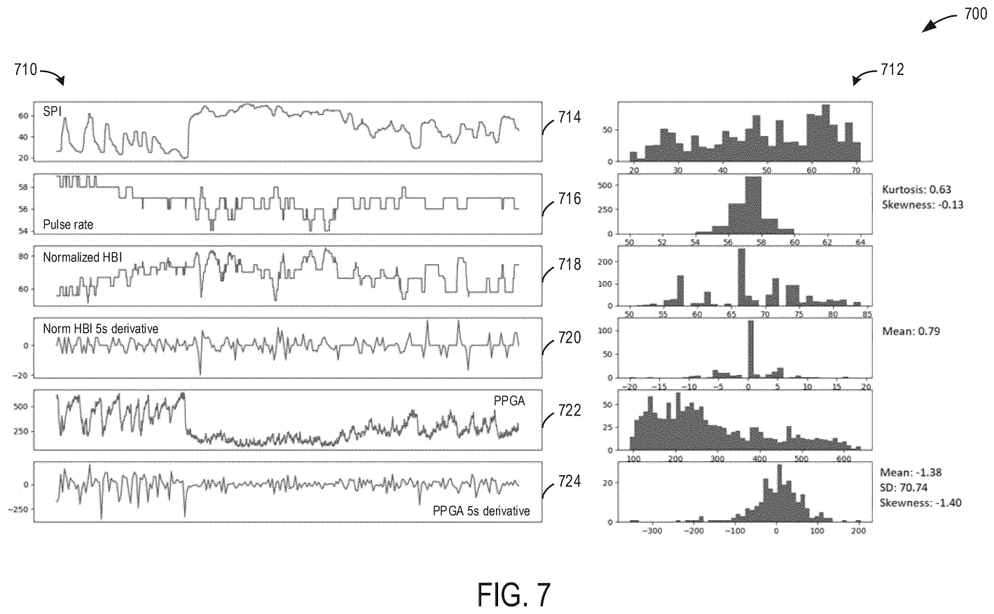

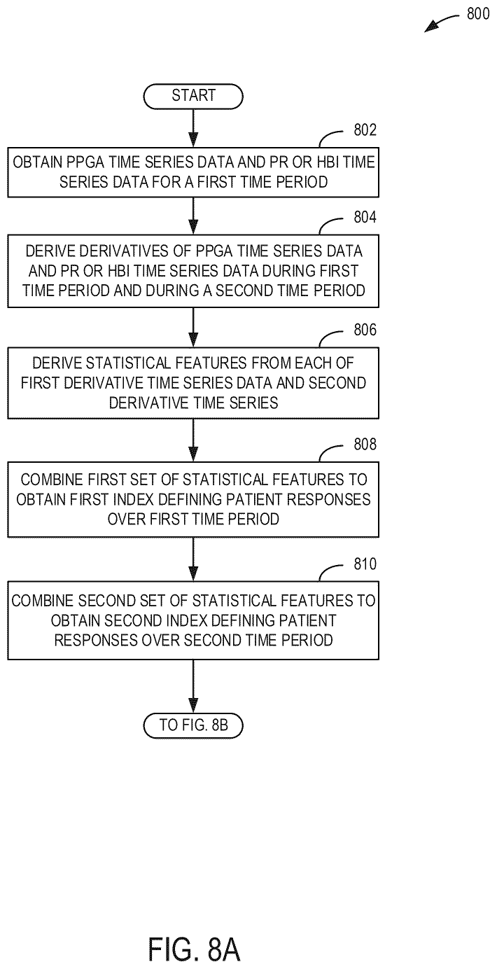

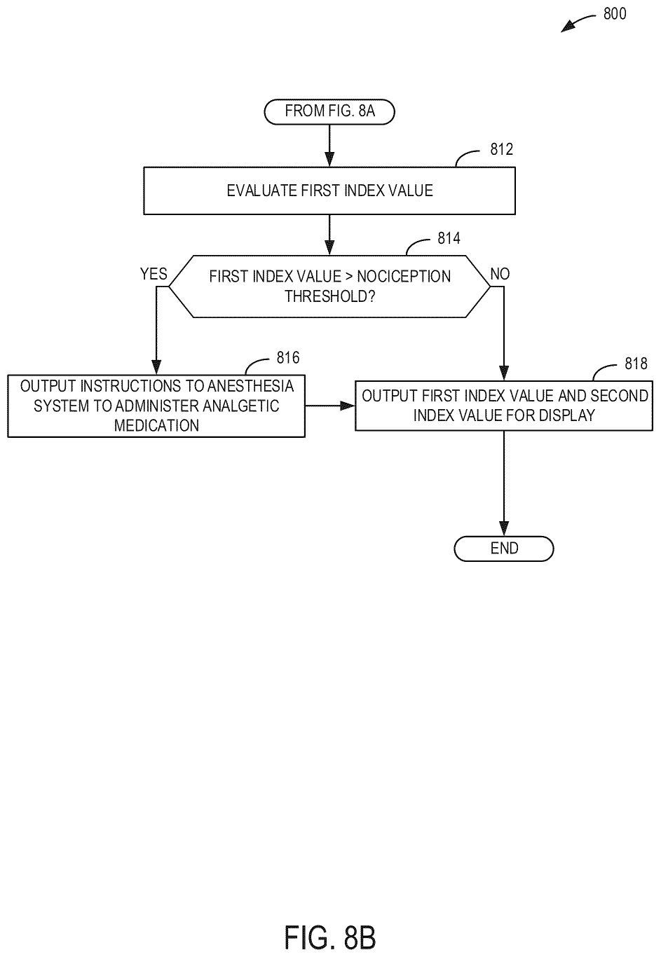

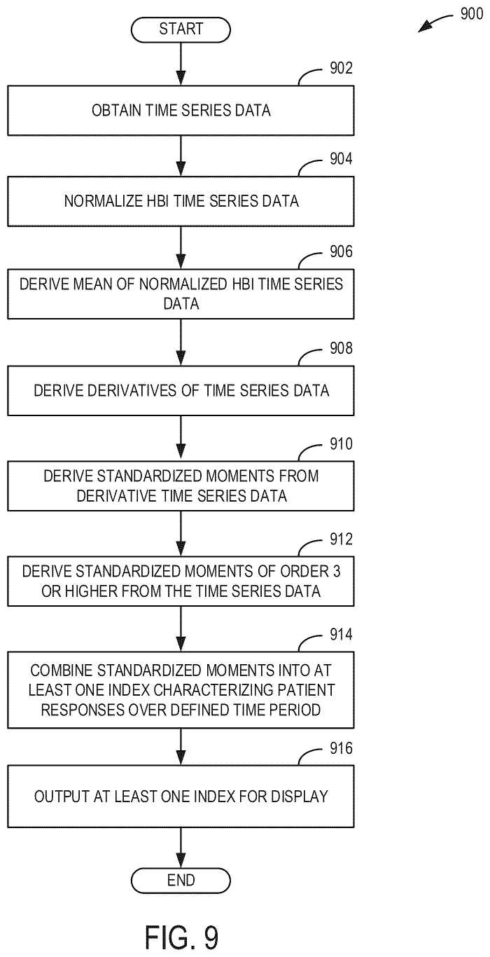

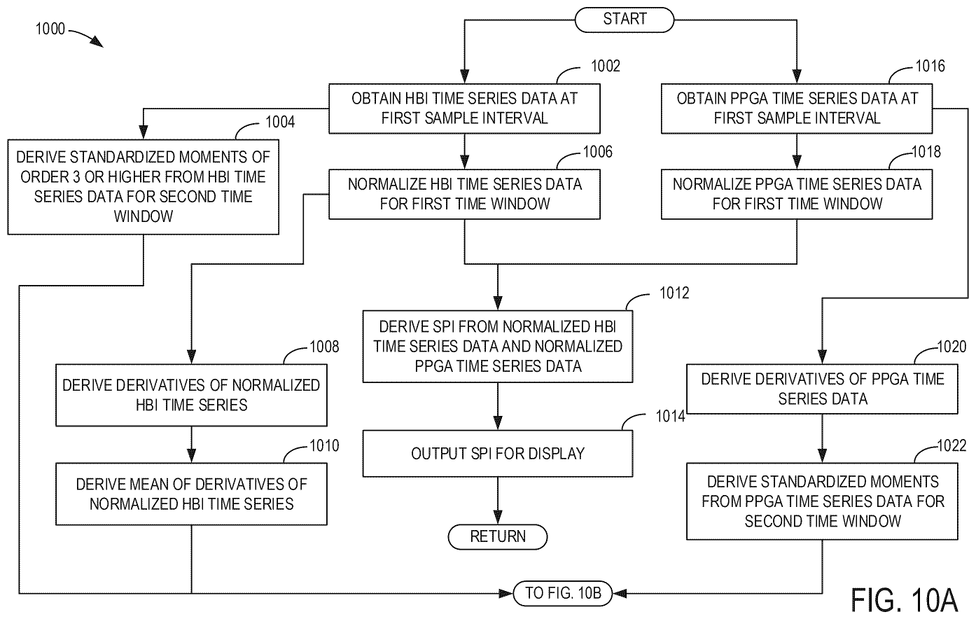

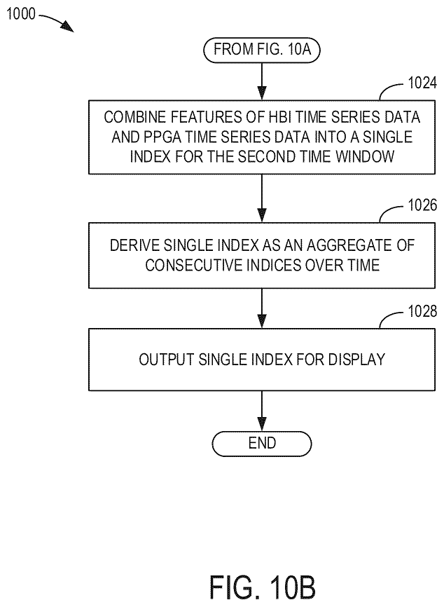

FIG. 1 shows an exemplary patient monitoring system, including a patient monitor configured to capture a hemodynamic response of a patient as photoplethysmogram signal data, a computing device configured to execute a method for quantifying a patient condition following completion of a surgical procedure using the photoplethysmogram signal data to generate a single index, and a display device configured to display the single index. FIG. 2 shows an example interface of the display device, including the single index quantifying a total stress index, a second index quantifying an instantaneous nociception response, and graphs of features used to calculate the first index and the second index. FIG. 3 shows a series of graphs illustrating performance of the method for quantifying the patient condition when the patient is not experiencing a hemodynamic response. Similarly, FIG. 4 shows a series of graphs illustrating performance of the method for quantifying the patient condition when the patient is experiencing a hemodynamic response. FIG. 5 shows a graph of Shapley additive explanation (SHAP) values of statistical features used to train the method for quantifying the patient condition, and FIG. 6 shows a graph of SHAP values of statistical features used to test the method for quantifying the patient condition. FIG. 7 shows a series of graphs illustrating hemodynamic response of a patient as photoplethysmogram signal data, and derivatives and statistical features thereof used to calculate the single index and the second index. FIGS. 8A-10B show flowcharts illustrating methods for quantifying the patient condition, where methods of FIGS. 9 and 10A-10B are variations of the method of FIGS. 8A and 8B. FIGS. 8A and 8B show a high-level flow chart of a method for quantifying the patient condition. FIG. 9 shows a high-level flow chart illustrating a method for generating a single index that characterized patient responses over a defined time period. FIGS. 10A and 10B show a flow chart illustrating a method for generating a first index defining a patient hemodynamic response over a first time period and generating a second index defining the patient hemodynamic response over a second time period.

FIG. 1 shows a block diagram illustrating an example patient monitoring system 100. The patient monitoring system 100 may be used in health care settings and comprises at least part of a patient monitor for monitoring plurality of physiological traits (e.g., heart rate, respiratory rate, blood sugar levels, blood cell count, blood oxygen levels, and so on) of a patient (not shown). To that end, the patient monitoring system 100 may include a plurality of patient monitoring devices 102, including a first monitoring device 104, a second monitoring device 106, and a third monitoring device 108. Each monitoring device of the plurality of patient monitoring devices 102 may be configured to monitor a different physiological trait of the patient. For example, one or more of the patient monitoring devices 102 may be configured obtain a hemodynamic response of a patient as photoplethysmogram signal data including a photoplethysmogram signal amplitude (PPGA) and/or a heartbeat interval (HBI). The patient monitoring devices may include, but are not limited to, an invasive and/or non-invasive blood pressure monitoring device, a pulse-oximetry monitoring device, a temperature monitoring device, a carbon dioxide monitoring device, a respiration monitoring device, a cardiac output monitoring device, and so on. The number of monitoring devices depicted is exemplary and non-limited, as it should be appreciated that in some embodiments the plurality of patient monitoring devices 102 may include a number of monitoring devices that is greater than three or less than three.

The patient monitoring system 100 further includes a computing device 112 communicatively coupled to each of the plurality of patient monitoring devices 102. The plurality of patient monitoring devices 102 provide measurements of physiological characteristics of a patient to the computing device 112. The computing device 112 includes a processor 114 and a non-transitory memory 116. The processor 114 is in electronic communication (e.g., communicatively connected) with the plurality of patient monitoring devices 102. As used herein, the term “electronic communication” may be defined to include both wired and wireless communications.

The processor 114 may control the one or more of the plurality of patient monitoring devices 102 to acquire data according to instructions stored on a memory of the processor 114 and/or the non-transitory memory 116. The various methods and processes described further herein may be stored as executable instructions in the non-transitory memory 116 of the computing device 112 in the patient monitoring system 100. The processor 114 may include a central processing unit (CPU), according to an embodiment. According to other embodiments, the processor 114 may include other electronic components capable of carrying out processing functions, such as a digital signal processor, a field-programmable gate array (FPGA), or a graphic board. According to other embodiments, the processor 114 may include multiple electronic components capable of carrying out processing functions. For example, the processor 114 may include two or more electronic components selected from a list of electronic components including: a central processor, a digital signal processor, a field-programmable gate array, and a graphic board. A patient condition quantifying module 118 may be stored as executable instructions in the non-transitory memory 116 that, when executed, cause the processor 114 to carry out methods for quantifying the patient condition of a patient, as described further herein with respect to FIGS. 8A-10B. Data (e.g., photoplethysmogram signal data captured by one or more of the plurality of patient monitoring devices 102) may be processed in real-time as data is received by the processor 114. For purposes of this disclosure, the term “real-time” is defined to include a procedure that is performed without an intentional delay (e.g., substantially at the time of occurrence).

The computing device 112 is also communicatively coupled to a display device 120. The display device 120 may be configured to display a single index that indicates a total nociception level of a patient, and a second index that indicates an instantaneous nociception response of the patient, as determined according to the methods described further herein. The patient monitoring system 100 may include multiple display devices that are communicatively coupled to the computing device 112 via a wired or a wireless connection, which may enable display of the one or more indices on more than one display device simultaneously. The processor 114 is also in electronic communication with the display device 120, and the processor 114 may process data captured by one or more of the plurality of patient monitoring devices 102 into outputs for display on the display device 120, as further described herein. Display device 120 may include one or more display devices utilizing virtually any type of technology. In some embodiments, display device 120 may comprise a computer monitor, and may display the single index, the second index, and one or more graphs showing photoplethysmogram signal data and/or statistical features derived therefrom.

Additionally, the patient monitoring system 100 may include a user input device 122 configured to control operation of the patient monitoring system 100. For example, the user input device 122 may be communicably coupled to one or more of the plurality of patient monitoring devices 102 and the display device 120 via the computing device 112. The user input device 122 may be used to control the input of patient data (e.g., patient medical history), to change a scanning parameter (e.g., a sampling frequency) of one or more of the plurality of patient monitoring devices 102, to change a display parameter of the display device 120, and the like. The user input device 122 may include one or more of a rotary element, a mouse, a keyboard, a trackball, hard keys linked to specific actions, soft keys that may be configured to control different functions, and a graphical user input device displayed on a display device 120. In some embodiments, the display device 120 may include a touch-sensitive display, and thus, the display device 120 may be included in the user input device 122.

In some embodiments, the patient monitoring system 100 may further include an anesthesia system 124 configured to administer pain medication to the patient. The anesthesia system 124 may be communicably coupled to the computing device 112. As further described herein, the computing device 112 may be configured with instructions to execute a method that comprises evaluating the single index and, in response to a value of the single index exceeding a nociception threshold, outputting instructions to the anesthesia system 124 to adjust administration of analgesic medication.

Further, the components of the patient monitoring system 100 may be coupled to one another to form a single structure, may be separate but located within a common room, or may be remotely located with respect to one another. For example, one or more of the modules described herein may operate in a data server that has a distinct and remote location with respect to other components of the patient monitoring system 100, such as the first monitoring device 104 and the user input device 122. Optionally, the patient monitoring system 100 may be a unitary system that is capable of being moved (e.g., portably) from room to room. For example, the patient monitoring system 100 may include wheels or may be transported on a cart, or may comprise a handheld device. In various embodiments of the present disclosure, one or more components of the patient monitoring system 100 may be included in a portable, handheld device. For example, the display device 120 and the user input device 122 may be integrated into an exterior surface of the handheld device, which may further contain the processor 114 and the non-transitory memory 116 therein.

FIG. 2 shows an example interface 200 of a patient monitoring system in a predictive view 202. The patient monitoring system may be the patient monitoring system 100 of FIG. 1, and thus the interface 200 may be an example of the display device 120. In FIG. 2, the interface 200 in the predictive view 202 is shown as being displayed after completion of a surgical procedure and conclusion of a method for quantifying a patient condition, as described with respect to FIGS. 8A-10B. The predictive view 202 may additionally or alternatively be displayed during the surgical procedure, which may enable a clinician to take preventive actions to patient post-operative condition. Alternatively, the predictive view 202 may be displayed automatically in response to completion of the surgical procedure, for example, when photoplethysmogram signal data is no longer being received from one or more patient monitors (e.g., the patient monitoring devices 102). In another example, the predictive view 202 may be displayed in response to user input via a user input device (e.g., the user input device 122) indicating the surgical procedure has ended and/or quantification of a patient condition is requested. In further examples, the predictive view 202 may be automatically displayed upon admittance of the patient to the PACU.

The predictive view 202 includes display of a single index 204 that defines an overall nociception response by a patient during a surgical procedure, and may be used to quantify a patient condition following conclusion of the surgical procedure. The single index 204 may additionally be referred to herein as “a first index” and/or “a total stress index”. As further described herein, the single index 204 is generated based on a hemodynamic response detected within a defined time period, where the defined time period is a non-fixed length, continuously increasing window of time that increases until the conclusion of the surgical procedure. The hemodynamic response is measured by one or more patient monitors (e.g., the patient monitoring devices 102), and defines a patient stress response to surgical nociceptive stimulus. For example, the hemodynamic response comprises photoplethysmogram signal data captured by a pulse oximeter. The value of the single index may not directly correspond to photoplethysmogram signal data captured during the surgical procedure, and is instead an aggregate value of statistical features of HBI time series data, statistical features of derivatives of PPGA time series data, and a mean of derivatives of normalized HBI time series data.

As further described herein with respect to FIGS. 5-10B, statistical features used to calculate the first index may include: a standard deviation of derivatives of PPGA, skewness of derivatives of PPGA, and a mean of derivatives of PPGA. The PPGA provides information about blood volume changes in the microvascular bed of tissue of a patient. As described above, the PPGA may be obtained using a patient monitor, such as a pulse oximeter, by illuminating the skin of a patient and measuring changes in light absorption. Each cardiac cycle of the patient appears as a peak (e.g., a peak of a PPG graph). The peak has an amplitude—the PPGA—that may be used to monitor heart rate and cardiac cycle, respiration, depth of anesthesia, hypo- and hypervolemia, and blood pressure. The standard deviation of derivatives of PPGA calculated from PPGA data of a single patient during a surgical procedure may indicate dispersion of the PPGA (e.g., of peak amplitudes). This dispersion may indicate changes in one or more of the patient's heart rate and cardiac cycle, respiration, depth of anesthesia, hypo- and hypervolemia, and blood pressure during the surgical procedure. For example, if the patient is not sufficiently anesthetized for the surgical procedure, the PPGA may depress (e.g., vasoconstriction of peripheral arteries may cause a sudden drop in PPG amplitude) in response to an incision performed during the surgical procedure, due to a sympathetic nervous response of the patient to the incision. In addition to the standard deviation of derivatives of PPGA, the skewness of derivatives of the PPGA and the mean of derivatives of the PPGA may be used to quantify instances of PPGA drops, which may indicate instances of nociceptive (e.g., sympathetic nervous system) responses throughout the surgical procedure. As further described herein, these statistical features of the PPGA may be weighted and used to calculate the first index quantifying the patient condition.

Statistical features used to calculate the first index may further include a pulse rate (PR) kurtosis, PR skewness, and a mean of derivatives of normalized HBI. The PR kurtosis illustrates distribution of PR data between a distribution's center and tails, and PR skewness characterizes an asymmetry of data distribution. A patient's pulse rate may be relatively unchanged for some of the duration of the surgical procedure, and may increase and/or decrease during other parts of the surgical procedure. Changes in the patient's pulse rate during the surgical procedure may indicate a nociceptive response. PR skewness may illustrate whether outlier PRs occur in the data captured during the surgical procedure, and the PR kurtosis may illustrate an amount of outlier PRs in the PR data. For example, many instances of high PRs (e.g., positive skewness and high kurtosis) may indicate a nociception response that may negatively affect the patient condition following the surgical procedure. The HBI illustrates a time between waves of the cardiac cycle, and the mean of derivatives of normalized HBI may summarize HBIs of the patient during the surgical procedure in a single value. This single value may be used as another indicator of patient condition. For example, if the mean of derivatives of normalized HBI is relatively high for a patient (e.g., compared to a pre-surgical procedure value), sympathetic nervous system of the patient may have experienced one or more nociceptive responses during the surgical procedure. These statistical features may be weighted and used to calculate the first index quantifying the patient condition, as further described herein.

Calculation of the first index using the above described statistical features may include applying weights to each of the features. For example, the standard deviation of the derivatives of the PPGA may have a first, highest weight, followed sequentially by the kurtosis PR having a second, lower weight, the skewness PR having a third, lower weight, the skewness of derivatives of PPGA having a fourth, lower weight, the mean of derivatives of the PPGA having a fifth, lower weight, and the mean of derivatives of normalized HBI having a sixth, lowest weight. In other embodiments for quantifying the patient condition, one or more of the statistical features may have different relative weights. Further, additional or alternative statistical features may be used by the methods described herein.

The first index may be calculated using weighted values of each of the above described statistical features in a logistic regression model. A logistic regression model models log-odds (e.g., a probability function) of an event as a linear combination of independent variables. For example, the logistic regression model is used by the method herein to predict a patient condition following conclusion of a surgical procedure. Specifically, an example logistic regression model is trained to predict POP of a patient, as described herein with respect to FIG. 5. Briefly, the logistic regression model is trained based on an importance of different statistical features in predicting POP. Described another way, the logistic regression model is trained such that an output (e.g., the first index) of the logistic regression model reflects an extent to which a weighted sum of statistical features influences POP. A trained logistic regression model is implemented by the method described herein to quantify a patient condition by applying weights to values of the statistical features of a nociception response of the patient during a surgical procedure to generate the first index.

In some embodiments, the single index may have a range of zero to ten, similar to the numeric rating scale (NRS), where a relatively high value (e.g., nine, on a scale of zero to ten) indicates high patient stress and potential for high POP, and a relatively low value (e.g., one, on the scale of zero to ten) indicates low patient stress and low likelihood of POP. In some embodiments, such as shown in FIG. 2, the single index 204 may be displayed as a numeric value. In other embodiments, the single index may be displayed as a color gradient that similarly corresponds to the NRS. For example, a relatively high value that indicates high patient stress may be displayed as a red color, and a relatively low value that indicates low patient stress may be displayed as a green color. The single index may further be displayed as a combination of a numeric value and a color, or other display methods that indicate a relative patient stress and POP level (e.g., a graphic or image, a scale, and so on) without departing from the scope of the disclosure.

The defined time period in which data is captured that is used to calculate the single index is a non-fixed length, continuously increasing window of time that increases until the conclusion of the surgical procedure, and includes at least part of a time of the surgical procedure. For example, the defined time period may include all of the surgical procedure, including pre-operative events, or may start at the commencement of operation. The defined time period does not have a fixed length, thus the method described herein may be used to quantify a patient condition following surgical procedures having a variety of lengths (e.g., more complex surgical procedures may take more time compared to relatively simple surgical procedures). As the defined time period is a continuously increasing window of time, the method described herein for quantifying the patient condition may be executed until receiving indication that the surgical procedure has ended, as further described herein.

A first value of the first index may be calculated using data captured during the defined time period. For example, the hemodynamic response measured by one or more patient monitors is sampled every second for a twenty-minute time window, and statistical features are calculated following conclusion of the twenty-minute time window to identify the first value of the first index. In this context, the statistical features are measures characterizing data values and distribution, such as mean, standard deviation, skewness, kurtosis and percentiles, for example. The value of the first index may be updated using additional data captured after conclusion of the first twenty-minute window. The hemodynamic response of a patient may be sampled for another defined time period (e.g., a twenty-minute window that is sampled at a rate of 1 Hz). In some embodiments, data from a second defined time period may be used to calculate a second value of the first index. This process may be continued to capture multiple values of the first index from consecutive time periods. The first index may be updated by aggregating a present value (n) of the first index and a subsequent value (n+1) of the first index. The first index (e.g., the total value of the first index) may be updated at a first rate, such as once per minute. In another embodiment, the value of the first index is updated by re-deriving statistical features from a dataset including hemodynamic response data captured during a first defined time period and one or more subsequent defined time periods (e.g., where each defined time period is twenty minutes).

As further described herein, a logistic regression model used in part to calculate the first index uses a sliding, fixed-length twenty-minute window that slides one minute at a time, such that the logistic regression model is updated at one-minute intervals with hemodynamic response data captured in the immediately preceding one minute. An aggregate value calculated using the output of the logistic regression model is thus updated at one-minute intervals, in some embodiments. Intervals at which the output of the logistic regression model is updated may be shorter than one-minute intervals in some embodiments. However, in the herein described systems and methods, updating the output of the logistic regression model at one-minute intervals may decrease computational demand on the processor, compared to updating the output at shorter intervals (e.g., more frequently). The aggregate measure is calculated using a window of time with a continuously growing duration, as further described herein.

The predictive view 202 further includes display of a second index 206 that indicates an instantaneous nociception response of the patient during a fixed-length, sliding time window within the defined time period. The second index 206 may additionally be referred to herein as “a SPI”, “an instantaneous nociception response”, and/or “an instantaneous stress index”. The fixed time period is shorter than and included within the defined time period. For example, the fixed time period may be five minutes or less, and may be updated at a rate of one second, such that the second index 206 is generated/updated at a frequency of 1 Hz. Thus, the second index 206 may be continuously updated during the defined time period, where the first index 204 is calculated following completion of the defined time period (e.g., 20 minutes) as a summarizing indicator of a total nociception response throughout the defined time period. The second index 206 may have a range of zero to ten, similar to the numeric rating scale (NRS), as described above.

Initial values of the first index 204 and the second index 206 may be displayed in the predictive view 202 at different times. Data collected by PPG monitoring during a first duration (e.g., a first five minutes, from start) of the defined time period (e.g., 20 minutes) may be used to normalize the second index 206. While the second index 206 is being normalized during the first duration, a first value of the second index 206 may be displayed. A first value of the first index 204 may not be displayed during the first duration, and instead a message may be displayed in the position of the first index 204. For example, a text-based message (e.g., “learning”) may be displayed in the place of the first index 204 to indicate the method is still collecting data used to generate the first index 204. Thus, the second index 206 may initially be displayed prior to display of the first index 204.

As further described herein with respect to FIGS. 5-10B, the first index 204 and the second index 206 are calculated, using different algorithms, from statistical features of photoplethysmogram signal data. Briefly, the first index 204 is calculated as an aggregate value of statistical features of the HBI time series data, statistical features of derivatives of the PPGA time series data, and a mean of derivatives of normalized HBI time series data during the first time period. The second index 206 is calculated as a combination of normalized HBI time series data and normalized PPGA time series data during the second time period for a single patient, captured using a patient monitor, such as a pulse oximeter as described with respect to FIG. 1. In some embodiments, the predictive view 202 further includes a series of graphs 208 that illustrates photoplethysmogram signal data and statistical features derived therefrom that are used to calculate the first index 204 and the second index 206. The series of graphs 208 may include a first series of graphs 210 including one or more of: a first graph 214 showing values of the second index 206 over time (e.g., during the defined time period), a second graph 216 of a pulse rate, a third graph 218 of a normalized heartbeat interval, a fourth graph 220 of a derivative of the normalized heartbeat interval (HBI) at a first frequency (e.g., five seconds), a fifth graph 222 of a PPGA, and a sixth graph 224 of a derivative of the PPGA at the first frequency. Each of the first series of graphs 210 may have the same time scale along the x-axis. The series of graphs 208 may also include a second series of graphs 212 that show histograms of a respective graph of the first series of graphs 210 (e.g., directly to the left of) as a bar graph. The histograms shown in the second series of graphs 212 show data distribution represented by higher order statistical moments (e.g., standard deviation, skewness, kurtosis). The series of graphs 208 further includes summarizing statistics of the pulse rate (e.g., kurtosis and skewness), the derivative of the normalized heartbeat interval at the first frequency (e.g., a mean value), and the derivative of the PPGA at the first frequency (e.g., a mean value, a standard deviation, and skewness). The summarizing statistics may be referred to herein as statistical features, and may be used to calculate the first index. Display of data used to calculate the first index 204 and the second index 206 may provide additional information which may be used by a medical provider to understand the values of the first index 204 and the second index 206, as well as changes in the patient nociception level throughout the surgical procedure.

In some embodiments, display of the series of graphs 208 may be optional. For example, a default display (e.g., a predictive view that is generated and output for display automatically in response to calculation of the first index) may include the first index 204 and the second index 206. As further described herein, information presented in the series of graphs 208 may not be visually recognizable by a user as indicating patient hemodynamic response during the surgical procedure. Thus, the information presented in the series of graphs 208 may not be of interest to the user and may not assist the user in quantifying the patient condition. By not displaying the series of graphs 208 and displaying the first index 204 and the second index 206, the predictive view includes a limited list of information most relevant to a user to avoid the user having to navigate through a series of menus, windows, etc. to see the various graphs/data that the indexes are based on. In some embodiments, one or more of the series of graphs 208 may be displayed in the perspective view in response to the computing device receiving a user input that selects the first index and/or the second index. In this way, the series of graphs 208 may be displayed in response to user input, instead of automatically, which may reduce processing demands, power consumption, and network traffic by avoiding the retrieval/generation of the graphs and rendering of the graphs when the graphs are not desired.

FIG. 3 shows a series of graphs 300 illustrating performance of the method for quantifying the patient condition following completion of a surgical procedure. The series of graphs 300 represent photoplethysmogram signal data captured from a single patient (e.g., via one or more patient monitors) not experiencing a hemodynamic response. A first graph 302 of the series of graphs 300 shows a first trend line 312 graphing state entropy (SE) over time, and a second trend line 314 graphing response entropy (RE) over time. SE and RE may be additional patient nociception responses that are used to quantify the patient condition, in some embodiments. SE and RE may also be used for automatic identification of the defined time period, e.g., indicating/predicting anesthesia onset and offset time points. For example, the collection of PPGA and PR/HBI time series data may reset or start after the SE and RE values drop at approximately 10 minutes and the patient is likely anesthetized. Correspondingly, an end of surgery time stamp may be automatically set to occur approximately at 210 minutes, where the SE and RE starts to incline. SE is less than or equal to RE for the duration of the first graph 302. A second graph 304 shows a trend line 316 graphing SPI (e.g., the second index) over time. The second graph 304 further includes a first shaded area 318 representing normalized pulse rate values, and a second shaded area 320 representing normalized PPGA values. A third graph 306 shows a logistic regression 322 (e.g., an output of the logistic regression model) calculated from the statistical features of the first graph 302 and the second graph 304. The third graph 306 also includes a nociception threshold 350, above which the patient may experience POP and below which the patient may not experience POP, as further described herein. The nociception threshold may be a configurable value. In the example of FIG. 3, the nociception threshold is 0.5, as shown in the third graph 306 and a fourth graph 308. The fourth graph 308 shows an aggregate measure 324 calculated from the logistic regression 322 of the third graph 306. The aggregate measure 324 is a graphical representation of the first index (e.g., the single index) that defines an overall nociception response by a patient during a surgical procedure and predicts post-operative pain following conclusion of the surgical procedure. The fourth graph 308 further includes the nociception threshold 350, and standard deviation plots 326.

A dataset used to generate the series of graphs 300 is an example dataset wherein, for a majority of the time, the patient did not have a hemodynamic response. Between 25 minutes and 50 minutes, the third graph 306 shows the logistic regression 322 briefly exceeding the nociception threshold 350. However, the aggregate measure 324 (e.g., the first index), including standard deviation plots 326 thereof, does not exceed the nociception threshold 350. The aggregate measure 324 is below the nociception threshold 350 in the fourth graph 308, thus the method for quantifying the patient condition may output (e.g., at the single index 204 of FIG. 2) a single index value predicting the patient will not experience POP.

FIG. 4 shows a series of graphs 400 illustrating performance of the method for quantifying the patient condition. The series of graphs 400 represent photoplethysmogram signal data captured from a single patient (e.g., via one or more patient monitor) experiencing a hemodynamic response. A first graph 402 of the series of graphs 400 shows a first trend line 412 graphing SE over time, and a second trend line 414 graphing response entropy RE over time. SE is less than or equal to RE for the duration of the first graph 402. A second graph 404 shows a trend line 416 graphing SPI (e.g., the second index) over time. The second graph 404 further includes a first shaded area 418 representing normalized pulse rate values, and a second shaded area 420 representing normalize PPGA values. A third graph 406 shows a logistic regression 422 (e.g., an output of the logistic regression model) calculated from the statistical features of the first graph 402 and the second graph 404. The third graph 406 also includes a nociception threshold 450, above which the patient may experience POP and below which the patient may not experience POP, as further described herein. The nociception threshold 450 may be a configurable value, for example, the value may be different for patients having different characteristics (e.g., age, sex, and so on). In the example of FIG. 4, the nociception threshold is 0.5, as shown in the third graph 406 and the fourth graph 408. A fourth graph 408 shows an aggregate measure 424 calculated from the logistic regression 422 of the third graph 406. The aggregate measure 424 is a graphical representation of the first index that defines an overall nociception response by a patient during a surgical procedure and predicts post-operative pain following conclusion of the surgical procedure. The fourth graph 408 further includes the nociception threshold 450, and standard deviation plots 426.

A dataset used to generate the series of graphs 400 is an example dataset wherein the patient experienced a hemodynamic response. Except for a brief moment between 15 minutes and 50 minutes, and following approximately 225 minutes, the third graph 406 shows the logistic regression 422 exceeding the nociception threshold 450. Similarly, the aggregate measure 424 (e.g., the first index), including standard deviation plots 426 thereof, exceed the nociception threshold 450 starting at approximately 25 minutes. In some embodiments, as further described herein with respect to FIGS. 8A-10B, the method may further include outputting instructions to administer analgesic medication in response to the first index exceeding the nociception threshold. This may account for the decrease in logistic regression trend line at approximately 225 minutes, as further described herein. The aggregate measure 424 (e.g., the first index) is greater than the nociception threshold 450 in the fourth graph 408 for a majority of the fourth graph 408, thus the method for quantifying the patient condition may output (e.g., at the single index 204 of FIG. 2) a single index value predicting the patient may experience POP. The first index may thus be smoother and more consistent than the second index, as the first index is an aggregate value of current and past data, and thus changes more slowly than the second index. As a result, if analgesic medicine is administered/adjusted automatically, using the first index to adjust administration/adjustment may result in less rapid changes to medicine administration (e.g., compared to using the second index), which may enable more efficient management of automatic pain administration (e.g., may reduce overuse of analgesic medication).

FIGS. 5 and 6 show graphs illustrating Shapley additive explanation (SHAP) values of statistical features used to train the method for quantifying the patient condition, and values of statistical features used when implementing the method for quantifying the patient condition, respectively. Briefly, a SHAP value shows an influence of each variable on the logistic regression model output. As described herein, the logistic regression model is the algorithm used to quantify the patient condition, based on hemodynamic response of a patient (e.g., photoplethysmogram signal data and statistical features thereof). In the examples of FIGS. 5 and 6, the logistic regression model is trained to predict POP of a patient using the statistical features described herein. In other embodiments of the method for quantifying a patient condition following conclusion of a surgical procedure using nociceptive response data captured during the surgical procedure, the logistic regression model may be trained to predict other characteristics of the patient condition, such as patient comfort. The data shown in FIG. 5 illustrates an impact of each statistical feature in a trained patient condition quantification algorithm (e.g., as described with respect to the methods of FIGS. 8A-10B). A graph of SHAP values illustrates feature importance. FIGS. 5 and 6 as described herein demonstrate that features used by the method described herein for quantifying a patient condition have statistical importance across large datasets (e.g., multiple patients).

A graph 500 of FIG. 5 and a graph 600 of FIG. 6 are shown as beeswarm plots that, in addition to showing relative importance of statistical features, shows relationships of the features with the predicted outcome (e.g., the first index). Each of the graph 500 and the graph 600 includes data points from the following statistical features: a standard deviation (Std) of derivatives (der) of PPGA captured at a five second interval, pulse rate (PR) kurtosis, PR skewness, skewness of derivatives of PPGA captured at a five second interval, a mean of derivatives of PPGA captured at a five second interval, and a mean of derivatives of normalized (norm) HBI captured at a five second interval. With reference to the above mentioned statistical features, “captured at a five second interval” is to be understood as PPGA or HBI data used to calculate the respective statistical feature is captured at a five second interval. Each point on the graph 500 and the graph 600 represent a feature value of a single patient within a twenty-minute time window (e.g., of a time period having a greater duration). The graph 500 and the graph 600 each represent data from multiple patients. Dense clusters of data show that many patients have the same level of the given feature value. For example, std PPGA 5 sec der does not show data points near zero, thus this feature may be used as a quantifier of patient condition. In other embodiments for quantifying the patient condition, additional or alternative statistical features may be used by the method, as described with respect to FIGS. 8A-10B.

The statistical features are arranged along a vertical axis in ascending order of mean absolute SHAP values for the entire data set. For example, Std PPGA 5 sec der has the highest mean absolute SHAP values, and mean PR norm 5 sec der has the lowest mean absolute SHAP values in both the graph 500 and the graph 600. In the beeswarm plot, for each statistical feature, each instance of the dataset appears as its own point. The points are distributed horizontally along the x-axis according to their SHAP values. In places where there is a high density of SHAP values, points are stacked vertically. Examining how the SHAP values are distributed reveals how a variable (e.g., statistical feature) may influence the logistic regression model's predictions.

A feature value bar 502 is shown along the vertical axis in greyscale herein, though it is to be understood that a more detailed view of the graph 500 and the graph 600 may be shown in color. The color bar corresponds to raw values of the statistical features for each instance. In FIG. 5 and FIG. 6, darker color of a data point indicates a value of a point of the feature is relatively high, and lighter color of the data point indicates a value of a point of the feature is relatively low. Examining color distribution horizontally along the x-axis for each variable provides insight into the general relationship between a statistical feature's raw value and its SHAP values.

As briefly described above with respect to FIG. 2, the logistic regression model may be trained to generate the first index from statistical features of the patient nociception response measured during the surgical procedure. The logistic regression model is trained based on an importance of different statistical features in predicting POP, as shown in the graph 500. Through feature selection, the six features described with respect to FIGS. 5 and 6 were identified as a combination of statistical features that may be easily obtained, measured, and/or calculated, and the identified statistical features were subsequently included in the logistic regression model. During the logistic regression model training phase, these features were assigned varying weights. The input features are ordered in the SHAP plots based on their importance, e.g., the extent to which each feature influences the logistic regression model output on average. The feature with the greatest influence on the first index value generated by the logistic regression model is positioned at the top. An influence of each statistical feature may be determined based on a type of data captured by the respective statistical feature. For example, the standard deviation of the derivative of the PPGA captured at a five second interval (e.g., a first statistical feature) has a higher impact on the logistic regression model output than the mean of the PPGA derivative captured at a five second interval (e.g., a second statistical feature). The first statistical feature illustrates deviation width of the PPGA derivatives, and the second statistical feature illustrates mean change of the PPGA throughout data collection (e.g., throughout the surgical procedure). The value of the first index is thus influence by both values of the nociception response and how those values change over the course of patient monitoring. This influence may be present in the first index, and may not be present in a shorter time period used to calculate the second index.

In this way, the logistic regression model is trained to generate an index value of the single index (e.g., the first index 204) based on weighted values of statistical features derived from nociception data captured during the surgical procedure. For example, as described with respect to FIGS. 5 and 6, the first index may be generated using a logistic regression model that uses a standard deviation of the derivative of the PPGA captured at a five second interval, a PR kurtosis, a PR skewness, a skewness of a PPGA derivative captured at a five second interval, a mean PPGA derivative captured at a five second interval, and a mean of normalized HBI derivative captured at a five second interval. The logistic regression model may include a series of weights that are applied to each of the statistical features listed above.

For example, the standard deviation of the derivatives of the PPGA may have a first, highest weight, followed sequentially by the kurtosis PR having a second, lower weight, the skewness PR having a third, lower weight, the skewness of derivatives of PPGA having a fourth, lower weight, the mean of derivatives of the PPGA having a fifth, lower weight, and the mean of derivatives of normalized HBI having a sixth, lowest weight. In other embodiments for quantifying the patient condition, one or more of the statistical features may have different relative weights. Also, non-linear models such as Random Forest or gradient-boosted trees can be used instead of linear logistic regression model. The patient condition is thus quantified as a single value.

As described above, FIG. 6 shows the graph 600 of SHAP values of features used to test the method for quantifying the patient condition, where the method is trained according to SHAP values as described with respect to FIG. 5. Distribution of data points in the graph 600 is similar to distribution of points in the graph 500, and ordering of the statistical features along the vertical axis is the same. This indicates that the logistic regression model is trained to have influences of the statistical features as described with respect to FIG. 5.

FIG. 7 shows a series of graphs 700 illustrating hemodynamic response of a patient as photoplethysmogram signal data, and derivatives and statistical features thereof. The series of graphs 700 includes the same types of graphs as are included in the series of graphs 208 of FIG. 2, generated from a different dataset (e.g., captured from a different patient and/or during a different surgical procedure). The series of graphs 700 may include a first series of graphs 710 including one or more of: a first graph 714 showing values of the second index over time (e.g., during the defined time period), a second graph 716 of a pulse rate, a third graph 718 of a normalized heartbeat interval (HBI), a fourth graph 720 of a derivative of the normalized heartbeat interval at a first frequency (e.g., five second), a fifth graph 722 of a PPGA, and a sixth graph 724 of a derivative of the PPGA at the first frequency. Each of the first series of graphs 710 may have the same time scale along the x-axis. The series of graphs 700 may also include a second series of graphs 712 that show histogram of a respective graph of the first series of graphs 710 (e.g., directly to the left of) as a bar graph. The histograms shown in the second series of graphs 712 show how higher order statistical moments (e.g., standard deviation, skewness, kurtosis) represent data distribution. The series of graphs 700 further includes summarizing statistics of the pulse rate (e.g., kurtosis and skewness), the derivative of the normalized heartbeat interval at the first frequency (e.g., a mean value), and the derivative of the PPGA at the first frequency (e.g., a mean value, a standard deviation, and skewness). Display of data used to calculate the first index and the second index may provide additional information which may be used by a medical provider to understand the values of the first index and the second index, as well as changes in the patient nociceptive responses throughout the surgical procedure. Thus, as described above, the logistic regression model used to determine the first index may calculate the first index using values of statistical features. For example, the standard deviation of the derivatives of the PPGA is 70.74, the kurtosis PR is 0.63, the skewness PR is −0.13, the skewness of derivatives of PPGA is −1.40, the mean of derivatives of the PPGA is −1.38, and the derivative of a mean, normalized HBI captured at a five second interval is 0.79.

FIGS. 8A and 8B show a high-level flow chart of a method 800 for generating an indicator of total surgical stress experienced by a patient (e.g., the single index/the first index) which may be used to quantify a patient condition following completion of a surgical procedure, and an indicator of instantaneous nociception responses (e.g., the second index/the SPI), according to an embodiment of the disclosure. Method 800 may be implemented by the computing device 112 of the patient monitoring system 100 of FIG. 1. Method 800 may be carried out according to instructions stored in non-transitory memory and executed by a processor, such as the non-transitory memory 116 and the processor 114 of FIG. 1. The method 800 is executed in part during a surgical procedure, and in part following conclusion of the surgical procedure. Some operations of the method 800 may be executed simultaneously, while others may be executed in a stepwise manner, as described herein. The method 800 is a high-level method, and further detail regarding generation of the first index and the second index is described with respect to FIGS. 9-10B.

At 802, PPGA time series data and PR or HBI time series data are obtained for a first time period. The first time period may be the duration of the surgical procedure, and obtaining the time series data at 802 may include initiating data collection (e.g., via the patient monitor), where data collection continues for the duration of the surgical procedure. In some embodiments, subsequent operations of the method 800 are executed as time series data is obtained, as described herein. As described above, the first time period is a non-fixed length, continuously increasing window of time that increases until the conclusion of the surgical procedure. For example, the first time period may be at least twenty minutes. Thus, obtaining PPGA time series data and PR or HBI time series data at operation 802 includes commencing data collection, wherein time series data is continuously collected for at least the duration of the surgical procedure, and during further execution of the method 800, as described herein.

At 804, derivatives of PPGA time series data and PR or HBI time series data are derived for time series data captured during the first time period and a second time period.

Derivatives of the PPGA time series data and the PR or HBI time series data (e.g., whichever is obtained at operation 802) are derived following completion of the first time period using all of the respective data captured during the first time period. The second time period is a duration within and shorter than the first time period. For example, the first time period may be twenty minutes, and the second time period may be one second within the first time period. Both the first time period and the second time period may be configurable, for example, based on the expected and/or actual duration of the surgical procedure. For example, in another embodiment, the first time period may be one hour and the second time period may be five seconds.

The second time period is a fixed-length, sliding window of time. Thus, derivatives of the PPGA time series data and the PR or HBI time series data derived for the second time period may be derived in real time using data of a present window of time. For example, when the first time period is at least twenty minutes, the second time period may be five minutes. Thus, the first time period includes at least four instances of the second time period. Derivatives of the PPGA time series data and the PR or HBI time series data derived for the first time period may not be derived in real time and are derived following conclusion of the first time period using all data of the first time period. While calculation of the first time index may use data from a larger time window compared to the second index, the first index may be calculated using statistical features of data captured during the first time period, which may reduce processing demand compared to calculations that use all of the data captured during the first time period, as further described herein.

At 806, statistical features are derived from derivatives of each of the first derivative time series data and the second derivative time series data calculated at operation 804. As briefly described with respect to FIGS. 2-7, the statistical features may be derived from a pulse rate, a derivative of the normalized heartbeat interval, and a derivative of PPGA. The statistical features describe characteristics of the data and assist in providing summary information about the patient's pain response (e.g., nociception response) throughout the surgical procedure, while simultaneously reducing a processing demand by decreasing an amount of data used in each subsequent operation of the method 800. In some embodiments, and as further described with respect to FIGS. 9-10, deriving statistical features of the PPGA time series data and PR or HBI time series data includes deriving standardized moments to normalize the time series data.

At 808, the first set of statistical features derived at 806 are combined to obtain a first index defining patient responses over the first time period and which may be used to predict a patient condition. The first set of statistical features may be combined using a prediction algorithm trained as described with respect to FIGS. 5-6. As described above, the first index may be obtained following completion of the surgical procedure.

In some embodiments, data collections may continue following completion of the surgical procedure, and the first index may be updated to continue providing information about the patient pain response. For example, the first index may be updated at a frequency of once per five minutes, where data collected in the immediately preceding five-minute time window is added to the data of the surgical procedure, and the first index is recalculated as described with respect to the method 800.

At 810, the second set of statistical features derived at 806 are combined to obtain a second index defining patient responses over the second time period, and predicting instantaneous nociception response. In some embodiments, the second index may be continuously updated, as durations of the second time period within the first time period pass. For example, the second index may be updated at a rate of one second (e.g., 1 Hz) or at a rate of once per five seconds (e.g., 0.2 Hz) using a set of statistical features derived as described above from an immediately preceding second time period.

At 812, a value of the first index is evaluated. For example, as described with respect to FIGS. 3 and 4, it is determined if the aggregate value (e.g., the first index) is greater than or less than the nociception threshold. Further, in some embodiments, evaluating the value of the first index may include identifying a potential severity of the nociception response as indicated by the first index. For example, when the value of the first index is zero, the patient may not experience a nociception response or POP. A severity of nociception response and potential POP may increase as the value of the first index increases from zero. At 814, if it is determined the first index value is greater than the nociception threshold, the first index indicates the user experienced a nociception response during the surgical procedure, and the patient may experience POP. For example, as described with respect to FIGS. 3-4, the nociception threshold may be 0.5. When the value of the first index is greater than the nociception threshold, the method proceeds to 816.

At 816, instructions are output to an anesthesia system to administer analgesic medication or adjust a dose of analgesic medication being administered to the patient. As described above, a value of the first index may indicate a severity of the nociception response. When the value of the first index exceeds the nociception threshold, it may be desirable to provide analgesic medication to the patient to relieve the nociception response. Outputting instructions to administer analgesic medication may include using evaluation of the first index value a performed at 812 to determine one or more of a dose, a type, and/or a frequency of analgesic medication delivery.

At 818, the first index and the second index are output for display on a display device. In some embodiments, the method 800 proceeds to operation 818 in response to determining the first index value is not greater than the nociception threshold. The patient may experience some nociceptive response, for example, as described with respect to FIG. 3, however, the overall nociceptive response of the patient may not be indicative of POP. Thus, instructions may not be provided to the anesthesia system to administer analgesic medication or change the dosage of currently-administered analgesic medication. As described with respect to FIG. 2, the first index and the second index may be displayed simultaneously. In some embodiments, for example, when the set of graphs 208 are displayed alongside the first index and the second index, a user may navigate to a time point within the duration of the surgical procedure by interacting with the user input device to select a time point in a graph of the set of graphs 208. In response, the second index value for the selected time point may be displayed alongside the first index, which may remain unchanged, as the first index quantifies a total nociception response for the first time period.

In further embodiments, the first index may be updated to continue providing information about the patient pain response. For example, following output of instructions to the anesthesia system to administer analgesic medication, the method may include updating the first index to monitor an efficiency of the analgesic medication in relieving the nociceptive response and POP.

FIG. 9 shows a high-level flow chart illustrating a method 900 for generating a single index (e.g., the first index) that characterizes patient responses over a defined time period, according to an embodiment of the disclosure. Method 900 may be a version of the method 800 of FIGS. 8A and 8B. Method 900 be implemented by the computing device 112 of the patient monitoring system 100 of FIG. 1. Method 800 may be carried out according to instructions stored in non-transitory memory and executed by a processor, such as the non-transitory memory 116 and the processor 114 of FIG. 1. The method 900 is executed in part during a surgical procedure, and in part following conclusion of the surgical procedure, (e.g., after admittance to a PACU.

At 902, the method 900 includes obtaining PPGA time series data and/or HBI time series data, collectively referred to herein as time series data. The PPGA time series data and/or HBI time series data may be obtained in real-time (e.g., as the data is captured) via one or more patient monitors. For example, as described with respect to FIGS. 1-2, a patient monitoring system may include a plurality of patient monitors, wherein one or more of the plurality of patient monitors is a pulse oximeter. The time series data may be continuously obtained for a duration of a defined time period. In some embodiments, the PPGA time series data and the HBI time series data may be obtained by the same patient monitor or by different patient monitors.

At 904, the method 900 includes normalizing HBI time series data obtained at operation 902. At 906, the method 900 includes deriving a mean value of normalized HBI time series data. Normalizing the HBI time series data and deriving the mean value of normalized HBI time series data may reduce data redundancy, identify statistical features of interest in the HBI time series data, and reduce an amount of data used to calculate the first index, which may reduce a processing and memory demand on the computing device compared to using the full, non-normalized dataset to calculate the first index.

At 908, the method 900 includes deriving derivatives of the time series data. This may include deriving derivatives of the PPGA time series data and/or the HBI time series data. Deriving derivatives of the HBI time series data may be performed on normalized HBI time series data.

At 910, the method 900 includes deriving standardized moments from the derivative time series data. In some embodiments, deriving standardized moments from the derivate time series data may include, for the HBI time series data, deriving a mean of the normalized HBI time series data. Deriving standardized moments from the derivative time series data normalizes the derivative time series data, such as by a power of the standard deviation, to render the standardized moments scale invariant. Described another way, the standardized moments are dimensionless numbers, which may further simplify processing methods and/or calculations used to generate the single index. Processing demand on may decrease, and a processing speed may increase, relative to a method for generating a single index using non-standardized time series data.

In some embodiments, the method 900 includes, at 912, deriving standardized moments of the degree 3 or higher from the time series data. For example, the method 900 may include deriving standardized moments of the order 3 or higher from the HBI time series data. Standardized moments of the HBI time series data may be performed without prior normalization of the HBI time series data. Further detail is described with respect to FIGS. 10A-10B.

At 914, the method 900 includes combining features into at least one index, wherein the at least one index characterizes patient responses over the defined time period. The method may be trained to assign different weights to different statistical features of the dataset to adjust influences of each variable on the logistic regression model output. Combining weighted statistical features into the at least one index (e.g., the first index) includes applying weights to each of the features in accordance with training of the logistic regression model, and combining values of the weighted features to provide the aggregate measure of statistical features that summarizes the patient nociception response and may be used to quantify the patient condition.

At 916, the method 900 includes outputting the at least one index, generated at operation 910, for display on a display device. As described with respect to FIGS. 2 and 8A-8B, outputting the at least one index may include outputting a numerical value (e.g., a predicted NRS pain score) and/or a color indicator illustrating an overall nociception response of the patient.

FIGS. 10A and 10B show a flow chart illustrating a method 1000 for generating a first index defining a patient hemodynamic response over a first time period and generating a second index defining the patient hemodynamic response over a second time period. Method 1000 may a version of the method 800 of FIGS. 8A and 8B. Method 1000 be implemented by the computing device 112 of the patient monitoring system 100 of FIG. 1. Method 1000 may be carried out according to instructions stored in non-transitory memory and executed by a processor, such as the non-transitory memory 116 and the processor 114 of FIG. 1. The method 1000 is executed in part during a surgical procedure, and in part following conclusion of the surgical procedure.

At 1002, the method 1000 include obtaining HBI time series data from a patient at a first sample interval, where the first sample interval is a fixed-length, sliding window. For example, data is collected at a frequency of one second, five seconds, five minutes, and so on, as is configured in response to user input. At 1016, the method 1000 includes obtaining PPGA time series data from the patient at the first sample interval. The HBI time series data and the PPGA time series data are simultaneously obtained. For example, in a first time window, a first data set including HBI time series data and PPGA time series data within the first time window is captured, and in a second time window that immediately follows and does not overlap with the first time window, a second data set including HBI time series data and PPGA time series data within the second time window is captured.

At 1006, the method 1000 includes normalizing HBI time series data for a first time window. At 1018, the method 1000 includes normalizing PPGA time series data for the first time window. The first time window may be, for example, five minutes. As described with respect to FIG. 9, normalizing HBI time series data and, similarly, normalizing PPGA time series data may reduce data redundancy in each of the HBI time series data and the PPGA time series data, which may reduce a processing and memory demand on the computing device compared to using non-normalized dataset to perform subsequent operations of the method 1000.

At 1012, the method 1000 includes deriving a SPI from normalized HBI time series data and normalized PPGA time series data. As described herein, SPI may be a dimensionless score based PPG analysis of pulse wave and heart beat intervals, and provides insight into a hemodynamic response of the patient to surgical nociceptive stimuli. At 1014, the method includes outputting the SPI for display at the display device. As described with respect to method 800 and method 900, the SPI may be continuously updated for each second time window, and the updated SPI may be output to the display as it is generated.

At 1004, the method 1000 includes deriving standardized moments of the order 3 or higher (e.g., including one or more of third moment skewness, fourth moment kurtosis, and the higher moments) from the HBI time series obtained at operation 1002 for a second time window. For example, the standardized moments may be derived from HBI time series data which has not been normalized. The second time window may be, for example, twenty minutes.

At 1008, the method 1000 includes deriving derivatives of the normalized HBI time series. For example, derivatives of the normalized HBI time series may be derived by subtracting a second HBI data element from a first HBI data element, where the second HBI data element is captured prior to the first HBI data element, and deriving a derivative from the difference. The second HBI data element may precede the first HBI data element by a second sample interval. For example, the second HBI data element may be captured five second earlier than the first HBI data element when the second sample interval is five seconds.

At 1010, the method 1000 includes deriving a mean of the derivatives of the normalized HBI time series data, as derived at operation 1008, for the second time window.

At 1020, the method 1000 includes deriving derivatives of the PPGA time series data captured at operation 1016. For example, the derivatives may be derived from PPGA time series data which has not been normalized. Derivatives of the PPGA time series data may be derived by subtracting a second PPGA data element from a first PPGA data element, where the second PPGA data element is captured prior to the first PPGA data element, and deriving a derivative from the difference. The second PPGA data element may precede the first PPGA data element by the second sample interval. For example, the second PPGA data element may be captured five second earlier than the first PPGA data element when the second sample interval is five seconds.

At 1022, the method 1000 includes deriving standardized moments of the derivatives of the PPGA time series data for the second time window. For example, standardized moments may be derived from the derivatives of the PPGA time series data derived at the operation 1020. As described above, the second time window may be twenty minutes.

At 1024, the method 1000 includes combining features of the HBI time series data and the PPGA time series data into a single index for the second time window. For example, standardized moments of the order 3 or higher of the HBI time series data (e.g., derived at operation 1004), a mean of the derivatives of normalized HBI time series data (e.g., derived at operation 1010), and standardized moments of the derivatives of the PPGA time series (e.g., derived at operation 1022) may be combined into the single index.

At 1026, the method 1000 includes deriving a total nociception response, where the total nociception response is an aggregate of consecutive indices over time. For example, operations of the method 1000 that are described as being performed for the second time window may be continuously performed for the duration of the second time window, such as at a rate of one second (e.g., 1 Hz), and the total nociception response may be updated on a second-by-second basis. At 1028, the method includes outputting the total nociception response for display on the display device. In this way, a first index (e.g., the total nociception response, derived at operation 1026) is derived, wherein the first index indicated an overall nociception response for the patient for a duration of a surgical procedure (e.g., the first time window) and a second index (e.g., the SPI, derived at operation 1012) is derived, wherein the second index indicates an instantaneous nociception response of the patient (e.g., within the second time window).

In the method described herein, the second index is a fixed-length moving time window, and an updated value of the second index is calculated from a duration of the fixed-length moving time window. For example, a first value of the second index is calculated from data captured during a first five-minute period, and a second, updated value of the second index is calculated from data captured during a second five-minute period, where the second five-minute period directly follows the first five-minute period with no gap in time there between. In some embodiments, the duration of the fixed length moving time window may be greater than or less than five minutes. For example, the duration may be one second, one minute, and so on. The value of the second index therefore provides a ‘snapshot’ insight into a patient pain condition in real time (e.g., with no intentional delay following data capture, such as when the duration is one second) or in near-real time (e.g., when the duration is five minutes), compared to the first index that provides a summary of the patient pain condition following an end of a surgical procedure (e.g., no further surgical events occurring and no further data is collected).

Another method for visualizing information about the patient pain condition in real time or in near-real time, and summarizing the patient condition following the end of the surgical procedure may include continuously updating a single patient pain value throughout a duration of, and following the end of, the surgical procedure. For example, after a first time period of a first duration of the surgical procedure, a value of a patient pain index may be calculated using data captured during the first time period. Similar to the method described above, the first duration may be one second, ten seconds, one minute, five minutes, and so on. In this method however, the patient pain index value may be updated during each subsequent time period of the first duration by recalculating the patient pain index to include data captured during the first time period and additional time periods of the first duration following the first time period. For example, a first value of the patient pain index may be calculated using data captured during a first time period with a duration of five minutes, and a second value of the patient pain index may be calculated using data captured during a second time period with a duration of five minutes and data captured during the first time period. In this example, the second value of the patient pain index is calculated using data captured during over a ten-minute duration. This method may be undesirable, however, as it includes constant and increasing demand on the processor to compute a single index value from a data set that is continuously increasing in size. In the method described herein with respect to FIGS. 8A-10B, calculating and updating the second index is comparatively less intensive on the processor, as the second index is calculated from a fixed-size data set to illustrate the patient pain condition in real time or near-real time. Therefore, calculation of the real time or near-real time patient pain index is more efficient when calculated as described herein. Additionally, the single value (e.g., the first index) described herein is calculated at the end of the surgical procedure from elements of a dataset including data captured throughout the surgical procedure. For example, when the second index is calculated at a frequency of once per five minutes and the surgical procedure has a duration of twenty minutes, the second index may be calculated and updated four times, and the first index may be calculated after twenty minutes. In some embodiments, the first index may be updated one or more times following an initial generation of the first index (e.g., after twenty minutes). For example, data may continue to be captured at the sampling frequency used to capture data and calculate the second index, and/or at a different sampling frequency, following conclusion of the surgical procedure. Continuing to capture data and update the first index may provide insight into post-operative patient recovery and changes in POP. Additionally, in embodiments where the method includes outputting instructions to an anesthesia system to administer analgesic medication following the first index exceeding the nociception threshold, updating the first index one or more times following administration of analgesic medication may provide insight into an efficiency of the analgesic medication.

Compared to the above described method where the single index value is computed from the continuously increasing data set (e.g., a large data set), the method described with respect to FIGS. 8A-10B is more efficient, as statistical features of the dataset are identified and used to calculated the first index. Thus, a technical effect of quantifying the patient condition as disclosed herein is that calculation of the first index may be more efficient for processing and memory usage, as relatively simple calculations may be performed on elements of the full dataset (e.g., calculating mean values, skewness, and so on) to identify statistical features, and the statistical features (e.g., a smaller dataset than the dataset including all data captured during the surgical procedure) are used to calculate the first index.

Additionally, the method described herein uses a high sampling frequency (e.g., between 1 Hz and per 5 minutes) to capture data and calculate the second index. Benefits of updating the second index at a high rate (e.g., equal to the high sampling frequency) include providing real time or near-real time information about a patient pain experience while not increasing processing demand to an undesirable level. In some embodiments, the sampling frequency and the rate at which the second index is updated may be adjustable, for example, in response to receiving user input. Additionally, the second index update rate may correspond to an efficiency of pain medication administer to a patient. For example, a pain medication may be known to take effect (e.g., begin to relieve pain of the patient) two minutes after administration of the pain medication by an anesthesia device. The sampling frequency may be adjusted to a frequency of two minutes, for example, so data is not recorded at a time prior to the pain medication having an effect (e.g., the patient condition is unchanged). The sampling frequency and effects of the pain medication may thus be on the same temporal scale. This may further reduce a processing and memory demand on the computing device executing the method.

The methods described herein may additionally provide a more accurate assessment of the patient pain condition, both during and following conclusion of the surgical procedure, compared to conventional methods that includes pain assessment performed by a user. Derivation and combinations of particular parameters from the data set captured during the surgical procedure may be time consuming and complex when performed by a user. Calculations to provide insight into the patient condition, including combined effects of time and optionally administering pain medication, may be quickly performed as described herein and thus provide real-time or near-real time insight into a patient condition that may be challenging to obtain when performed by a user.

In this way, both an instantaneous nociception response (e.g., the second index, via a SPI value) and an indicator of total surgical stress experienced by a patient (e.g., the first index, the single index) may be viewed on the same, single interface, which may allow an operator of an imaging system to simultaneously monitor patient condition via the live view and prepare for potential POP or other patient conditions, as predicted by the method.

The disclosure also provides support for a method for quantifying a patient response to surgery, comprising: generating a single index that quantifies a patient condition of a patient using a nociception response of the patient during a surgical procedure, where the nociception response includes a hemodynamic response of the patient detected within a non-fixed length, continuously increasing window of time that increases until conclusion of the surgical procedure. In a first example of the method, the method further comprises: evaluating the single index and, in response to a value of the single index exceeding a nociception threshold, outputting instructions to an anesthesia system to adjust administration of analgesic medication. In a second example of the method, optionally including the first example, the single index predicts a post-operative condition of the patient. In a third example of the method, optionally including one or both of the first and second examples, the hemodynamic response comprises photoplethysmogram signal data captured by a pulse oximeter. In a fourth example of the method, optionally including one or more or each of the first through third examples, a photoplethysmogram signal amplitude (PPGA) and/or a heartbeat interval (HBI) of the photoplethysmogram signal data are used to quantify the patient nociception response. In a fifth example of the method, optionally including one or more or each of the first through fourth examples, statistical features of the PPGA and/or the HBI are derived from data captured during a fixed-length, sliding window of a defined time period, and wherein the statistical features quantify hemodynamic responses over the defined time period. In a sixth example of the method, optionally including one or more or each of the first through fifth examples, generating the single index includes applying weights to each of the statistical features, and combining values of the weighted statistical features to provide an aggregate measure of statistical features that summarizes the patient nociception response. In a seventh example of the method, optionally including one or more or each of the first through sixth examples, a value of the single index is within a range of zero to ten. In an eighth example of the method, optionally including one or more or each of the first through seventh examples, the method further comprises: generating a second index indicating an instantaneous nociception response of the patient during a fixed-length, sliding window within a defined time period of the surgical procedure. In a ninth example of the method, optionally including one or more or each of the first through eighth examples, the method further comprises: updating the second index on a second-by-second basis.

The disclosure also provides support for a method, comprising: obtaining photoplethysmogram signal amplitude (PPGA) time series data, and pulse rate (PR) or heartbeat interval (HBI) time series data for a subject for a first time period, deriving derivatives of the PPGA time series data, and PR or HBI time series data captured during the first time period to obtain a first derivative time series for the first time period and during a second time period to obtain a second derivative time series for the second time period, wherein the second time period is included in and is shorter than the first time period, deriving statistical features from each of the first derivative time series and the second derivative time series to obtain a first set of statistical features and a second set of statistical features, respectively, combining the first set of statistical features to obtain a first index, the first index defining patient responses over the first time period, combining the second set of statistical features to obtain a second index, the second index defining patient responses over the second time period, and simultaneously outputting for display each of the first index and the second index. In a first example of the method, the first index is a total nociception response for the first time period and the second index is an instantaneous nociception response for the second time period. In a second example of the method, optionally including the first example, the first time period is at least twenty minutes and the second time period is five minutes. In a third example of the method, optionally including one or both of the first and second examples, deriving statistical features of the PPGA time series data and PR or HBI time series data includes deriving standardized moments. In a fourth example of the method, optionally including one or more or each of the first through third examples, obtaining HBI time series data includes normalizing HBI time series data, deriving derivatives of the HBI time series data includes deriving derivatives of normalized HBI time series data, and deriving a mean of normalized HBI time series data. In a fifth example of the method, optionally including one or more or each of the first through fourth examples, obtaining HBI time series data further includes deriving standardized moments of an order three or higher from the HBI time series data.

The disclosure also provides support for a patient monitoring system, comprising: a patient monitor, a display device, and a computing device communicably coupled to the patient monitor and to the display device, the computing device configured to: continuously obtain photoplethysmogram signal amplitude (PPGA) time series data and/or heartbeat interval (HBI) time series data during a first time period, generate a first index defining a patient hemodynamic response over a first duration of the first time period, where the first index is an aggregate value of statistical features of the HBI time series data, statistical features of derivatives of the PPGA time series data, and statistical features of derivatives of normalized HBI time series data during the first time period, generate a second index defining the patient hemodynamic response over a second time period of the first time period, the second time period shorter than and included in the first time period, where the second index is a combination of normalized HBI time series data and normalized PPGA time series data during the second time period, update the first index at a first rate for the first time period, update the second index after a duration of the second time period, and simultaneously output the first index and the second index to the display device in real-time. In a first example of the system, the patient monitor is a pulse oximeter. In a second example of the system, optionally including the first example, the display device is further configured to display, in real-time, photoplethysmogram signal data captured by the patient monitor. In a third example of the system, optionally including one or both of the first and second examples, the first rate at which the first index is updated is once per minute over the course of the first time period.

As used herein, the term “approximately” is construed to mean plus or minus five percent of the range unless otherwise specified.

As used herein, an element or step recited in the singular and preceded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

The following claims particularly point out certain combinations and sub-combinations regarded as novel and non-obvious. These claims may refer to “an” element or “a first” element or the equivalent thereof. Such claims should be understood to include incorporation of one or more such elements, neither requiring nor excluding two or more such elements. Other combinations and sub-combinations of the disclosed features, functions, elements, and/or properties may be claimed through amendment of the present claims or through presentation of new claims in this or a related application. Such claims, whether broader, narrower, equal, or different in scope to the original claims, also are regarded as included within the subject matter of the present disclosure.

1. A method for quantifying a patient response to surgery, comprising:
generating a single index that quantifies a patient condition of a patient using a nociception response of the patient during a surgical procedure, where the nociception response includes a hemodynamic response of the patient detected within a non-fixed length, continuously increasing window of time that increases until conclusion of the surgical procedure.

2. The method of claim 1, further comprising evaluating the single index and, in response to a value of the single index exceeding a nociception threshold, outputting instructions to an anesthesia system to adjust administration of analgesic medication.

3. The method of claim 1, wherein the single index predicts a post-operative condition of the patient.

4. The method of claim 1, wherein the hemodynamic response comprises photoplethysmogram signal data captured by a pulse oximeter.

5. The method of claim 4, wherein a photoplethysmogram signal amplitude (PPGA) and/or a heartbeat interval (HBI) of the photoplethysmogram signal data are used to quantify the nociception response.

6. The method of claim 5, wherein statistical features of the PPGA and/or the HBI are derived from data captured during a fixed-length, sliding window of a defined time period, and wherein the statistical features quantify hemodynamic responses over the defined time period.

7. The method of claim 6, wherein generating the single index includes applying weights to each of the statistical features, and combining values of the weighted statistical features to provide an aggregate measure of statistical features that summarizes the nociception response.

8. The method of claim 1, wherein a value of the single index is within a range of zero to ten.

9. The method of claim 1, further comprising generating a second index indicating an instantaneous nociception response of the patient during a fixed-length, sliding window of a defined time period during the surgical procedure.

10. The method of claim 9, further comprising updating the second index on a second-by-second basis.

11. A method, comprising:
obtaining photoplethysmogram signal amplitude (PPGA) time series data, and pulse rate (PR) or heartbeat interval (HBI) time series data for a subject for a first time period;
deriving derivatives of the PPGA time series data, and PR or HBI time series data captured during the first time period to obtain a first derivative time series for the first time period and during a second time period to obtain a second derivative time series for the second time period, wherein the second time period is included in and is shorter than the first time period;
deriving statistical features from each of the first derivative time series and the second derivative time series to obtain a first set of statistical features and a second set of statistical features, respectively;
combining the first set of statistical features to obtain a first index, the first index defining patient responses over the first time period;
combining the second set of statistical features to obtain a second index, the second index defining patient responses over the second time period; and
simultaneously outputting for display each of the first index and the second index.

12. The method of claim 11, wherein the first index is a total nociception response for the first time period and the second index is an instantaneous nociception response for the second time period.

13. The method of claim 11, wherein the first time period is at least twenty minutes and the second time period is five minutes.

14. The method of claim 11, wherein deriving statistical features of the PPGA time series data and PR or HBI time series data includes deriving standardized moments.

15. The method of claim 11, wherein obtaining HBI time series data includes normalizing HBI time series data, deriving derivatives of the HBI time series data includes deriving derivatives of normalized HBI time series data, and deriving a mean of normalized HBI time series data.

16. The method of claim 15, wherein obtaining HBI time series data further includes deriving standardized moments of an order three or higher from the HBI time series data.

17. A patient monitoring system, comprising:
a patient monitor;
a display device; and
a computing device communicably coupled to the patient monitor and to the display device, the computing device configured to:
continuously obtain photoplethysmogram signal amplitude (PPGA) time series data and/or heartbeat interval (HBI) time series data during a first time period;
generate a first index defining a patient hemodynamic response over a first duration of the first time period, where the first index is an aggregate value of statistical features of the HBI time series data, statistical features of derivatives of the PPGA time series data, and statistical features of derivatives of normalized HBI time series data during the first time period;
generate a second index defining the patient hemodynamic response over a second time period of the first time period, the second time period shorter than and included in the first time period, where the second index is a combination of normalized HBI time series data and normalized PPGA time series data during the second time period;
update the first index at a first rate for the first time period;
update the second index after a duration of the second time period; and
simultaneously output the first index and the second index to the display device in real-time.

18. The patient monitoring system of claim 17, wherein the patient monitor is a pulse oximeter.

19. The patient monitoring system of claim 17, wherein the display device is further configured to display, in real-time, photoplethysmogram signal data captured by the patient monitor.

20. The patient monitoring system of claim 17, wherein the first rate at which the first index is updated is once per minute over a course of the first time period.
